(19) United States
(12) Patent Application Publication
(10) Pub. No.: US 2017/0290882 A1
(43) Pub. Date: Oct. 12, 2017

(54) Specific Combined Therapy of Malignant Tumors with a Cytostatic and Its Modifier

(71) Applicant: AKTSYONERNOE OBSHCHESTVO "PEPTEK", Moscow (RU)

(72) Inventors: Tatyana Mikhaylovna Andronova, Moscow (RU); Raisa Ivanovna Yakubovskaya, Moscow (RU); Elena Romanovna Nemtsova, Moscow (RU); Evgeniya Ivanovna Nesterova, Moscow (RU)

(21) Appl. No.: 15/510,429

(22) PCT Filed: Nov. 23, 2015
(86) PCT No.: PCT/RU2015/000809

§ 371 Date: Mar. 10, 2017

(30) Foreign Application Priority Data
Apr. 16, 2015 (RU) 2015114103

(51) Int. Cl. A61K 38/14; A61K 33/24; A61K 9/00

(52) U.S. Cl. CPC A61K 38/14; A61K 9/0019; A61K 33/24

(57) ABSTRACT

The invention relates to medicine, particularly, to treating patients with malignant tumors using the combination of cytostatic and biotherapy.

Method for treating malignant hematological diseases or melanoma in subjects by applying one or more cytostatics impacting DNA in combination with N-acetyl-D-glucosaminyl-β-(1-4)-N-acetylmuramyl-L-alanyl-D-glutamic acid (GMDP-A) according to the following therapeutic sequence for subjects:
Intravenous injection of ¼to ½standard therapeutic dose of the cytostatic selected for this type of subjects;
Then, after cytostatic administration, the first injection of N-acetyl-D-glucosaminyl-β-(1-4)-N-acetylmuramyl-L-alanyl-D-glutamic acid (GMDP-A) in effective amount set forth for these subjects;
GMDP-A repeated injections in effective amount set forth for selected subjects.

Technical result of the alleged invention, which increases efficiency while treating malignant hematological diseases or melanoma, involves synergistic effect during the combined impact of a cytostatic and its immune modifier; this effect allows reduction of therapeutic dose of highly toxic cytostatics without reducing their anti-tumor effect. 1 independent claim, 3 dependent claims, 4 examples, 4 tables

The invention relates to medicine, particularly, to treating patients with malignant tumors using the combination of cytostatic and biotherapy.

Despite the undeniable achievements in modern oncology, increasing the efficiency of treatment methods for malignant tumors remains the extremely important problem.

Chemotherapy is the foreground treatment for patients with common malignant processes; however, it is often ineffective and highly toxic. This is largely due to the need to carry on chemotherapy against the unfavorable background of immune suppression induced by tumor process and worsened by the effect caused by the most cytostatics applied.

Immunotherapy as an independent type of cancer treatment is not very promising since immune drugs do not possess cytostatic activity in the vast majority of cases. Actually, only endogenic cytokines—interleukins and interferons—are used at the clinical practice to treat patients with certain forms of malignant tumors; however, the range of their anti-tumor effect is very limited while adverse reactions are severe enough (Deepika Narasimha, M D, et al., The International Journal of Targeted Therapies in Cancer; Immunotherapy in Advanced Melanoma; Jun. 3, 2012, p. 37-41).

The combination of cytostatic and biological therapy using immune drugs can significantly increase the treatment efficiency in certain cases.

The means of biological therapy used for such combined therapeutic sequences against malignant tumors comprise vaccines, synthetic peptides, homogeneous antibodies, cytokines and other products of modern biotechnology. Biotherapeutic drugs activate protective components of the immune system and initiate an antitumor effect. They also impact factors and mechanisms that control processes of cell proliferation and death thus resulting in a synergistic effect from combined application of cytostatic drug and a biological product in the therapeutic sequence (Gonzalez A B, Jimenez R B, Delgado P J R, et al. Biochemotherapy in the treatment of metastatic melanoma in selected patients. Clin Transl Oncol 2009; 11 (6): 382-6.); (Cohen D J, Hochster H S, Rationale for combining biotherapy in the treatment of advanced colon cancer. Gastrointest Cancer Res 2008; 2(3): 145-51). In other words, a biological drug acts simultaneously both as a modifier that increases efficiency of anti-cancer agent and a protector that prevents the body from an immune suppressive (immune toxic) effect of the cytostatic.

However, currently there is no systematic approach as how to select the triad “malignant tumor—cytostatic—biotherapeutic agent”. The current state of art/knowledge does not allow to extrapolate a priori positive experience in treatment of one tumor histological form by a pair “cytostatic—biotherapy agent” to another form. Since treatment efficiency depends on many factors: drug routes, doses, therapeutic sequence, etc., non-optimal choice of several parameters can result in the opposite effect while using immune drugs: tumor growth may be caused instead of its inhibition. This makes important the search for optimal combinations of chemotherapy and biotherapy as well as expanding the range of drugs for antitumor biotherapy, especially for tumors resistant to conventional cytostatics.

Modern medicines are high-tech products: vaccines, synthetic peptides, homogeneous antibodies, cytokines are very expensive at the current state of art. Their manufacturing is limited; and they are really not readily available to practicing clinicians. Therefore, the effect-oriented medicine pays attention to known immune modulators, which are commercially available or suitable for industrial production by conventional chemical methods while searching for cancer treatment by combines cytostatic and biotherapy.

The present invention aims at expending the range of immune drugs modulating (enhancing) effectiveness of cytostatics while treating immune-dependent cancer diseases, which are resistant to cytostatic therapy, using the combination of chemo—and biotherapy.

The prior art discloses the formulation containing cytostatic and muramyl peptide, which was successfully used to treat patients with recrudescent osteosarcoma by the combination of cytostatic and biotherapy (Nardin A, Lefebvre M L, Labroquére K, Faure O, Abastado J P. Liposomal muramyl tripeptide phosphatidylethanolamine: Targeting and activating macrophages for adjuvant treatment of osteosarcoma. Curr Cancer Drug Targets. 2006 March; 6(2):123-33). The patients undergone the combined treatment were featured by the longer recurrence-free and overall survival.

This clinical study was preceded by an experimental study of how efficient were the combinations of different cytostatics with muramyl peptide for dogs with spontaneous osteosarcoma and spleen hemangiosarcoma (MacEwen E. G., Kurzman I. D., Helfand S., Vail D., London C., Kisseberth W., Rosenthal R. C., et al. Current studies of liposome murarmyl tripeptide (CGP 19835A lipid) therapy for metastasis in spontaneous tumors: a progress review. J. Drug Target, 1994; 2(5): 391-6). During treatment of osteosarcomas in dogs, multiple intake of cisplatin was supplemented with repeated muramyl peptide intake. Dogs with hemangiosarcoma were treated with the combination of intravenous injection of doxorubicin with cyclophosphamide and repeated muramyl peptide intravenous intake. In both cases, muramyl peptide was a N-acetyl muramoyl-alanine-D-isoglutaminyl-alanyl-2-(1,2-dipalmitoyl)-sn-glycero-3-oforyl-ethylamide. The life time of animals with both tumors types treated with the combined chemotherapy and biotherapy was higher as compared with the animals treated with cytostatic monotherapy. However, intake of the immune drug has not resulted in dose decreasing for the chemotherapeutic agent that demonstrated an inadequate modulating (potentiating) effect of the applied muramyl peptide on cytostatic drugs.

The closest analog to the proposed invention is destruction of TNF-alpha-sensitive tumor cells with a formulation comprising TNF-alpha and muramyl peptide (RU 2209078 C1) added to cytostatic as a biotherapeutic agent. The proposed formulation modifies cytostatic efficiency and results in synergistic effect allowing the decrease of the cytostatic therapeutic dose (TD); i.e., it takes potentiating effect on the cytostatic. It was proposed to use N-acetyl-D-glucosaminyl-β-(1-4)-N-acetylmuramoyl-L-alanyl-D-isoglutamine (GMDP) and N-acetyl-D-glucosaminyl-β-(1-4)-N- acetylmuramoyl-L-alanyl-D-glutamic acid (GMDP-A) as muramyl peptides and cisplatin, doxorubicin or actinomycin D as cytostatics. According to this method, cytostatic, TNF-alpha and GMDP solutions with preset concentration were prepared separately and mixed then in certain proportions. Using cytolysis of tumor cells under the impact of the claimed formulation as a test, it was found that cytolysis of TNF-alpha-sensitive tumor cells ranged 72% to 100%. In experiments with animals (mice), the claimed formulation was injected intraperitoneally to animals with intraperitoneally-implanted Ehrlich ascites carcinoma. The mice survival rate was up to 100% at the cytostatic dose 4 times smaller than during the standard monotherapy with this drug. When exposed with the only chemotherapeutic agent or in combination with one of biotherapeutic components (TNF-alpha or muramyl peptide), no such efficiency was observed during the treatment.

Therapeutic effect was demonstrated to inhibit the growth of TNF-sensitive tumor cells in vivo and in vitro experiments for the formulation {GMDP: cysplatin: TNF- alpha=1:1.6: 0.0050} (component mass ratio). GMDP-A was not tested in the claimed formulation.

Application of TNF-alpha and GMDP in combined therapy is the disadvantage of the proposed method. TNF-alpha is a highly toxic compound (Nedospasov S. A., Kuprash D. V. Oncoimmunology: Certain fundamental problems of cancer immunotherapy. Molecular biology 2007; 41(2); 355-368) and may enhance immune suppression of a therapy subject with neoplastic disease. GMDP demonstrates a pyrogenic effect during intravenous injection that prevents its application in oncologic clinical practice. Probably, due just to these reasons, this study did not gain further traction.

The undoubted disadvantage of invention (RU 2209078 C1) was the fact that the authors have not considered potential ability of GMDP-A to form aggregative or covalent bonds with TNF-alpha in solutions reducing its effect. It was known that TNF-alpha covalent or aggregative derivatives may be prepared by bonding compounds via groups in its side amino-acid chains (RU 2076151). Cystein or histidin residues are the preferred sites in the TNF-alpha molecule to form its derivatives. Such covalent or aggregative derivatives would not yet perform their cytokine functions. Therefore, no data about application of GMDP-A in the proposed formulation in the examples of this invention leaves unproven the possibility of its use for combined therapy to treat tumor TNF-alpha—sensitive cells.

The problem solved by the alleged invention is to develop an efficient combined cytostatic and biotherapy to treat malignant hematological diseases or melanoma. A technical result of the alleged invention, which increases efficiency while treating malignant hematological diseases or melanoma, involves a synergistic effect during the combined impact of a cytostatic and its immune modifier; this effect allows reduction of therapeutic dose of highly toxic cytostatics without reducing their antitumor effect.

The specified result is achieved by treating malignant hematological diseases or melanoma in subjects by applying one or more cytostatics impacting DNA in combination with N-acetyl-D-glucosaminyl-β-(1-4)-N-acetylmuramyl-L-alanyl-D-glutamic acid (GMDP-A) according to the following therapeutic sequence for subjects:
Intravenous injection of ¼ to ½ standard therapeutic dose of the cytostatic selected for this type of subjects;
Then, after cytostatic administration, the first injection of N-acetyl-D-glucosaminyl-β-(1-4)-N-acetylmuramyl-L-alanyl-D-glutamic acid (GMDP-A) in effective amount set forth for these subjects;
GMDP-A repeated injections in effective amount set forth for selected subjects.

In this case, an animal or a human may be the subject of therapy during particular implementation of the invention; the first and subsequent injections are made subcutaneously; GMDP-A is injected at first in an hour after cytostatic administration, and repeated GMDP-A subcutaneous injections are made once a day within 4-20 days.

In the alleged invention, it was proposed at first to use N-acetyl-D-glucosaminyl-β-(1-4)-N-acetylmuramyl-L-alanyl-D-glutamic acid (GMDP-A) as an individual immune modifier of cytostatics, which impact mainly DNA, during the combined cytostatic and biotherapy for malignant hematological diseases or melanoma.

Using GMDP-A in tumor therapy with the aim to relieve the cytostatic immune toxicity and myelosuppression caused by it, the authors of the invention have revealed the previously unknown ability of N-acetyl-D-glucosaminyl-β-(1-4)-N-acetylmuramyl-L-alanyl-D-glutamic acid (GMDP-A) to potentiate the effect of cytostatics, which impact mainly DNA, while treating immune-dependent malignant hematological diseases and melanoma under certain dosage and therapeutic sequences. Therefore, despite the previous data about the possibility of GMDP-A application to inhibit the tumor cell growth (U.S. Pat. No. 4,395,399A; RU 96109376A; WO 9809989; EP 0722332B1; US 20071673555), the ability of GMDP-A to potentiate the effect of cytostatics, mainly impacting DNA, during the treatment of malignant hematological diseases and melanoma, was established for the first time.

GMDP-A may be produced in sufficient amount using technologically-efficient and relatively low-cost peptide synthesis methods described in U.S. Pat. No. 4,395,399. GMDP-A is apyrogenic in a wide range of concentrations, including potentially therapeutic. Preclinical studies of acute, chronic and specific toxicity of GMDP-A have shown no toxic action of this substance both at assumed therapeutic dose and at its five-fold excess (Report of the Institute of Immunology, Russian Academy of Medical Sciences, M., 1998; Report of the Russian Scientific Center for Security of Biologically Active Substances, Moscow region, Staraya Kupavna, 2014). Thus, GMDP-A may be used in clinical practice if a reasonable method exists for its application.

GMDP-A used in the present invention is injected parenterally, which can be done subcutaneously, intradermally or intramuscularly. Subcutaneous administration is preferable; it provides efficient drug interaction with target cells: dendritic cells, Langerhans cells and macrophages. Intra-tumoral injection (into tumor tissue) of GMDP-A is also possible.

Effective amount expressed as a single therapeutic dose is 0.002 mg/kg to 8.825 mg/kg in vivo (mice).

Dose per treatment course in vivo (mice) is 0.012 mg/kg to 185.300 mg/kg.

According to the proposed invention, GMDP-A for parenteral administration may be prepared as a pre-dozed sterile lyophilisates for preparation of injection solutions and as sterile solutions containing a suitable pharmaceutical carrier.

If a lyohilizate is used, saline solution, water for injections may be a solvent for GMDP-A substance as well as other ones usually used for this purpose.

In case of solutions for injection, aqueous carrier is preferable: saline solution (0.9% NaCl), glycin solution (0.3%) and similar known carriers may be used. Such solvents as propylene glycol, dimethyl sulfoxide, dimethyl formamide and various their mixtures may be also used apart from aqueous carriers. The solution may also contain suitable excipients, such as buffer substances, inorganic salts to achieve normal osmotic pressure, other substances to increase stability of GMDP-A solutions. Sodium and potassium salts (chloride or phosphate), sucrose, glucose, mannitol, sorbitol, protein hydrolysates, dextran, polyvinyl pyrrolidone, polyethylene glycol, disodium edentate may be the examples of such additives.

Cytostatics of different classes, preferably selected from the group of cytostatics mainly impacting DNA, may be used in combination with N-acetyl-D-glucosaminyl-β-(1-4)-N-acetylmuramyl-L-alanyl-D-glutamic acid (GMDP-A) modifier during the proposed specific combined therapy for malignant hematological diseases or melanoma. Thus, GMDP-A has potentiating effect on cyclophosphan (alkylating cytostatic), cisplatin (platinum compound), gemcitabine (an anti-metabolite) during treatment of malignant hematological diseases and melanoma. Their predominant effect on DNA cells is the common feature of these cytostatic agents.

Administration schedule for the proposed drugs for the claimed specific combined therapy is acceptable for clinical practice.

This technical solution has the following characteristic features:
Application of N-acetyl-D-glucosaminyl-β-(1-4)-N-acetylmuramyl-L-alanyl-D-glutamic acid (GMDP-A) as a cytostatic modifier during the specific combined therapy for malignant hematological diseases or melanoma;
Identification of an exclusive pair {a cytostatic drug mainly affecting DNA+GMDP-A} for the specific combined therapy for malignant hematological diseases and melanoma, which can achieve a synergistic effect allowing to decrease therapeutic dose of highly toxic cytostatic without reducing the effectiveness of its anti-tumor impact;
Mode of treatment of malignant hematological diseases and melanoma with combined cytostatic and biotherapy, which allows to implement the synergy effect caused by an exclusive pair (a cytostatic drug mainly affecting DNA+GMDP-A) and results in the lower therapeutic dose of highly toxic cytostatic agent without reducing the effectiveness of its anti-tumor impact.

We consider these characteristics as significant since their combination allows to obtain new and unexpected result—potentiating effect of GMDP-A on cytostatics mainly impacting DNA, increasing efficiency of cancer treatment, which is unpredictable in advance.

This result is caused by:

a) The ability of GMDP-A, which is tropic to NOD2 receptors, to activate non-specific and specific protection mechanisms of the human body by stimulating a large variety of immune responses. They include formation of cytokine cascades reducing immune-suppressive effect of tumors and cytostatics, on the one hand, and cytokine cascades enhancing antitumor protection, on the other hand;

b) Properly chosen specific combined cytostatic and biotherapy {a cytostatic drug mainly affecting DNA+GMDP-A}, sensitive to phenotype of malignant tumors and the cytostatic impact mechanism;

c) Properly chosen treatment mode, which takes into account the ability of GMDP-A, as many other immune drugs, to have a multi-directional effect on proliferation of tumor cells and their sensitivity to the cytostatic effect depending on its dose and therapeutic sequence.

The advantage of the alleged invention is the increase of the binary therapy efficiency by using GMDP-A as a relatively inexpensive, apyrogenic and non-toxic modifier for a cytostatic agent.

Another advantage of the proposed method of specific combined therapy is the fact that the relationship was established between phenotypic parameters of tumors (only malignant hematologic diseases or melanoma), the mechanism of cytostatic effect (mainly impacting DNA), the chemical structure of the modifier-muramyl peptide (only GMDP-A) and anti-tumor response. Thus, the proposed method contains the criteria, which may be used with maximum efficiency, for their “inclusion”/“exclusion” into/from the treatment protocol.

The GMDP-A ability to correct myelosuppression, which may be caused by the cytostatic used (WO9809989), may be an additional advantage of the proposed method.

### The Data Confirming the Invention Feasibility

The real implementation of the proposed specific combined therapy for malignant hematological diseases and melanoma by the effect of a cytostatic mainly affecting DNA and its modifier was illustrated by examples of the study carried out using animals with transplanted tumors.

The following tumor models were used to study the efficiency of the proposed specific combined therapy: P388 lymphoplastic leukemia (P388) and B16 melanoma (B16). They were transplanted on the right-side surface of the animal body.

Cyclophosphan, cisplatin and gemcitabine were used as cytostatics mainly affecting DNA.

GMDP-A was used as a cytostatic modifier as solution for injections or lyophilizates complying with pharmacopeia requirements.

The animals with tumors were treated in test groups at the early (24 hours after tumor inoculation) and late (5 days after tumor inoculation) terms of the tumor growth using a single cytostatic injection intravenously in a dose equal to ½ or ¼ of therapeutic one and various schedules for GMDP-A injection:
Primary subcutaneous injection of GMDP-A modifier in a single dose 3.75 mg/kg;
Repeated protracted subcutaneous administration of GMDP-A modifier according to different schedules: 4-fold, 9-fold and 20-fold, once per day, in a single dose of 3.75 mg/kg (course dose is 18.75 mg/kg, 37.5 mg/kg and 78.75 mg/kg, correspondingly). Dosage frequency depends upon the model (histological form of transplanted tumor).

To identify efficiency of the claimed combined therapy, independent medication was carried out as a control after tumor inoculation using one component of binary therapy-cytostatic drug or a modifier. Cytostatic was injected intravenously in amount of ½ or ¼ of therapeutic dose once after 24 hours or 5 days after tumor inoculation; GMDP-A modifier was administered subcutaneously in a single dose of 3.75 mg/kg 5-fold from the 1^{st} day to the 5^{th} day or from the 5^{th} day to the 9^{th} day, or 10-fold from the 1^{st} day to the 10^{th} day or from the 5^{th} day to the 14^{th} day, or 21-fold from the 1^{st} day to the 21^{st} day after tumor inoculation.

Animals without exposure were the common control for all groups.

Anti-tumor efficiency of the claimed combined therapy and comparative monotherapy using only a cytostatic or only GMDP-A modifier was assessed by the criteria commonly used in experimental oncology: tumor volume (TV), tumor growth inhibition (TGI), increase of the animal life time (ILT), tumor metastasis frequency (MF), metastasis inhibition rate (MI).

The following values TGI>70%; ILT>50%; MI>75% were efficiency criteria of anti-tumor and anti-metastasis properties.

### EXAMPLE 1

### Efficiency Assessment for the Combined Therapy “DDP+GMDP-A” in the P388 Lymphoplastic Leukemia Model

Efficiency of the combined therapy—chemotherapy with cisplatin cytostatic (DDP) and bioherapy with GMDP-A was assessed on P388 lymphoplastic leukemia model inoculated to mice BDF₁, females, subcutaneously on the right-side body surface.

1 mg/ml GMDP-A solution for subcutaneous injection was prepared by dissolving GMDP-A lyophilisate in water for injections.

GMDP-A was administered to mice subcutaneously as a single dose of 3.75 mg/kg every day with different schedules:
Five-fold (total dose is 18.75 mg/kg) on the 1^{st} day to the 5^{th} day or on the 5^{th} day to the 9^{th} day after tumor inoculation;
Ten-fold (total dose is 37.5 mg/kg) on the 1^{st} day to the 10^{th} day or on the 5^{th} day to the 14^{th} day after tumor inoculation;
Twenty-one-fold (total dose is 78.75 mg/kg) on the 1^{st} day to the 21^{st} st day after tumor inoculation.

DDP (Cisplatin, Cisplatin Teva trade mark, manufacturer is Teva Pharmaceutical Industries Ltd., Israel, fabricated at Pharmachemie B.V., the Netherlands) was administered intravenously as a single dose ma 4 mg/kg (½ of therapeutic dose).

Experimental results are shown in Table 1.

Example 1 demonstrated that addition of GMDP-A biotherapy to DDP chemotherapy (injecting ½ therapeutic dose) credibly increased treatment efficiency by TGI and MI for the P388 lymphoplastic leukemia model. Moreover, modifying effect of GMDP-A relatively DDP in therapeutically ineffective dose (½ therapeutic dose) was the same for all used administration schedules at the early treatment start (24 hours after tumor inoculation).

Efficiency of combined therapy somewhat decreased at the late treatment start (on the 5^{th} day after tumor inoculation); however, it remains credibly better at the 10-fold administration of GMDP-A as compared to chemotherapy using only cytostatic (Table 1).

### EXAMPLE 2

### Efficiency Assessment for the Binary Therapy “DDP+GMDP-A” in the B16 Melanoma Model

Efficiency of the combined therapy—chemotherapy with DDP cytostatic and biotherapy with GMDP-A was assessed on B16 melanoma model inoculated to BDF₁ mice, females, subcutaneously on the right-side body surface.

1 mg/ml GMDP-A solution for subcutaneous injection was prepared by dissolving GMDP-A lyophilisate in water for injections.

GMDP-A was administered to mice subcutaneously as a single dose of 3.75 mg/kg every day with different schedules:

Five-fold (total dose is 18.75 mg/kg) on the 1^{st} day to the 5^{th} day or on the 5^{th} day to the 9^{th} day after tumor inoculation;

Ten-fold (total dose is 37.5 mg/kg) on the 1^{st} day to the 10^{th} day or on the 5^{th} day to the 14^{th} day after tumor inoculation;

DDP (Cisplatin, Cisplatin Teva trade mark, manufacturer is Teva Pharmaceutical Industries Ltd., Israel, fabricated at Pharmachemie B.V., the Netherlands) was administered intravenously as a single dose of 4 mg/kg (½ of therapeutic dose).

Experimental results are shown in Table 2.

Efficiency of the combined impact due to chemo-and biotherapy in this model is similar to that in the model P388. The most effective schedule is 10-fold GMDP-A injection after DDP chemotherapy (½ therapeutic dose) at the early treatment start (24 hours after tumor inoculation) and the 5-fold injection at the late one (on the 5^{th} day after tumor inoculation).

As it can be seen from the data given in examples 1 and 2, the efficiency of GMDP-A for individual use (without chemotherapy) did not reach a biologically significant level.

### EXAMPLE 3

### Efficiency Assessment for the Combined Therapy “Chemotherapy (Different Cytostatics)+GMDP-A” in the P388 Lymphoplastic Leukemia Model

Comparative study was carried out of the GMDP-A modifying effect relatively different cytostatic agents: cisplatin (DDP), gemzar and cyclophosphan (CP), in the P388 lymphoplastic leukemia model. Efficiency of combined therapy with cytostatics and GMDP-A was assessed while using DDP in amount of ½ therapeutic dose, gemzar as ½ therapeutic dose and CP in amount of ¼ therapeutic dose. Treatment was started on the early term of tumor growth (in 24 hours after inoculation); GMDP-A was injected during 21 days in a daily dose of 3.75 mg/kg. The drug was administered subcutaneously in 2 areas: in the tumor growth area (over the tumor) or in symmetry area (on the opposite side of the tumor node).

The following commercially-available drugs were used: Cyclophosphan Endoxan® trade mark, manufactured by Baxter Oncology GmbH, Germany; Cisplatin, Cisplatin Teva trade mark, manufacturer is Teva Pharmaceutical Industries Ltd., Israel (fabricated at Pharmachemie B. V., the Netherlands); Gemcitabine, Gemcitar trade mark, manufactured by CJSC Biocad, Russia.

1 mg/ml GMDP-A solution for subcutaneous injection was prepared by dissolving GMDP-A lyophilisate in water for injections.

Results are given in Table 3.

As it can be seen from the results of Example 3, GMDP-A efficiently modifies therapeutic effect of all used chemotherapeutic agents by TGI, ILT and MI indicators; moreover, modifying degree differed unreliably when injecting in different areas. This fact indirectly confirms the GMDP-A immune-modulating effect, which appears not only after injection close to the tumor nidus but also remotely after injection in the opposite side.

The degree of the GMDP-A modifying effect depends upon the cytostatic used and appears only while using the cytostatics mainly impacting DNA cells while their impact on RNA is minimal (hence, resulting in obtaining minimum amount of defective RNA that prevents synthesis of specific proteins, which implement potentiating effect of GMDP-A).

### EXAMPLE 4

### Comparative Efficiency Assessment for the Combined Therapy “Chemotherapy DDP+GMDP-A” in the P388 Lymphoplastic Leukemia Model While Using GMDP-A Lyophilisate and “GMDP-A, 1 mg/ml Solution for Subcutaneous Injection” Final Pharmaceutical Form

Therapeutic efficiency of GMDP-A lyophilisate and the GMDP-A final pharmaceutical form was compared in the model of P338 lymphoplastic leukemia solid version at the early treatment start (in 24 hour after subcutaneous transplantation of tumor material) as GMDP-A monotherapy (lyophilisate or the final pharmaceutical form) as well as combined administration with DDP.

Experimental conditions for the lyophilisate were described in Example 1.

As a final pharmaceutical form, “GMDP-A, 1 mg/ml solution for subcutaneous injection” sterile solutions were used. They contained GMDP-A and the following auxiliary substances and carriers in pharmaceutically-accepted amounts: sorbitol, disodium edeteate, propylene glycol, water.

As it may be seen from the data given in Table 4, GMDP-A lyophilisate and final pharmaceutical form demonstrate the similar effect: they result in reliable increase of anti-tumor efficiency by all indicators in combination with a cytostatic. TGI for groups with combined treatment exceeds by approx. 30% the same parameter in the group with DDP treatment for all observation periods. ILT is 44%, 37% and 22% in groups “GMDP-A lyophilisate+DDP”, “GMDP-A, 1 mg/ml solution for subcutaneous injection+DDP” and DDP, while metastasis inhibition in these groups is 82%, 79% and 32%, correspondingly.

Thus, GMDP-A lyophilisate and the GMDP-A final pharmaceutical form have the similar modifying impact relatively DDP in the P338 lymphoplastic leukemia model.

TABLE 1
GMDP-A effect on the primary tumor growth, metastasis and DDP therapeutic effect in mice with P-388

| | | Tumor volume, mm³ tumor growth inhibition (TGI), % | | | | | Average life | | |
|---|---|---|---|---|---|---|---|---|---|
| Group | | tumor growth day | | | | | time, days | Metastasis | |
| number | Therapeutic sequence | 7 | 9 | 12 | 14 | 19 | ILT, % | MF, % | MI, % |

Treatment at the early tumor progress (the 1^{st} day of tumor growth)

| | | | | | | | | | |
|---|---|---|---|---|---|---|---|---|---|
| 1 | 5-fold GMDP-A | 45 ± 2 | 124 ± 7 | 204 ± 54 | 257 ± 24 | 318 ± 42 | 22 ± 1.2 | n/d | n/d |
| | | 51 | 36 | 6 | 16 | 6 | 21 | | |
| 2 | 10-fold GMDP-A | 66.6 ± 6 | 108.6 ± 14.2 | 187.3 ± 27.4 | 245.3 ± 30.5 | 566.9 ± 50.2 | 24 ± 0.5 | 100 | 247 ± 24.7 |
| | | 37 | 24 | 4 | 9 | 11 | 0 | | 10 |
| 3 | 21-fold GMDP-A | 73.5 ± 4.8 | 120.6 ± 12.5 | 159.0 ± 14.7 | 200.7 ± 17.3 | 432.1 ± 67.9 | 23 ± 1.6 | 100 | 215 ± 42.2 |
| | | 30 | 16 | 18 | 25 | 32 | −4 | | 22 |
| 4 | single DDP + 5-fold | 1 ± 1 | 7 ± 7 | 19 ± 14 | 23 ± 21 | 67 ± 29 | 25 ± 3.1 | n/d | n/d |
| | GMDP-A | 99 | 96 | 91 | 92 | 80 | 39 | | |
| 5 | single DDP + 10-fold | 3.4 ± 0.9 | 6.4 ± 2.6 | 23.2 ± 5.3 | 41.2 ± 6.3 | 192.3 ± 25.4 | 25 ± 0.6 | 100 | 33 ± 2.8 |
| | GMDP-A | 97 | 96 | 88 | 85 | 70 | 4 | | 88 |
| 6 | single DDP + 21-fold | 5.4 ± 1.2 | 6 ± 3 | 10.7 ± 4.8 | 40.4 ± 5.6 | 140.0 ± 17.1 | 27 ± 0.3 | 100 | 55.8 ± 18.9 |
| | GMDP-A | 95 | 96 | 94 | 85 | 78 | 13 | | 80 |
| 7 | DDP | 37.9 ± 4.5 | 67.5 ± 7.7 | 76.7 ± 7.7 | 155.3 ± 12 | 374.1 ± 28.4 | 23 ± 0.7 | 100 | 141 ± 13.9 |
| | | 64 | 53 | 61 | 42 | 41 | −4 | | 49 |

Treatment at the late tumor progress (the 5^{th} day of tumor growth)

| | | | | | | | | | |
|---|---|---|---|---|---|---|---|---|---|
| 8 | 5-fold GMDP-A | 63 ± 12 | 107 ± 18 | 172 ± 51 | 257 ± 53 | 302 ± 103 | 20 ± 0.9 | n/d | n/d |
| | | 31 | 45 | 21 | 16 | 11 | 7 | | |
| 9 | 10-fold GMDP-A | 86.5 ± 5.9 | 105 ± 9 | 163.8 ± 13.5 | 242.4 ± 18.3 | 510.1 ± 46.6 | 24 ± 0.8 | 100 | 253 ± 41.9 |
| | | 18 | 26 | 16 | 10 | 20 | 0 | | 8 |
| 10 | single DDP + 5-fold | 36 ± 5 | 61 ± 6 | 81 ± 16 | 106 ± 24 | 180 ± 78 | 25 ± 1.1 | n/d | n/d |
| | GMDP-A | 61 | 69 | 63 | 66 | 47 | 40 | | |
| 11 | single DDP + 10-fold | 62.4 ± 7.5 | 28.6 ± 4.4 | 25.3 ± 3.5 | 55.6 ± 6.6 | 188.8 ± 21.9 | 25 ± 2.7 | 100 | 47 ± 8.4 |
| | GMDP-A | 41 | 80 | 87 | 79 | 70 | 4.0 | | 83 |
| 12 | DDP | 75.4 ± 4.8 | 65.7 ± 6.2 | 95.9 ± 8.4 | 149.3 ± 11.6 | 448.9 ± 33.2 | 24 ± 0.6 | 100 | 136 ± 3.3 |
| | | 28 | 54 | 51 | 44 | 30 | 0 | | 50 |

Control groups

| | | | | | | | | | |
|---|---|---|---|---|---|---|---|---|---|
| 13 | 24 hours →21-fold water | 102.7 ± 5.9 | 146.6 ± 8.3 | 201.2 ± 11.5 | 274.6 ± 24.2 | 638.0 ± 41.2 | 24.0 ± 0.7 | 100 | 263.0 ± 23.2 |
| | for injections | 3 | −3 | −4 | −2 | 0 | 0 | | 4 |
| 14 | 5 days →10-fold water | 104.5 ± 5.4 | 147.3 ± 10.3 | 204.8 ± 14.5 | 278.0 ± 22.6 | 632.8 ± 43.0 | 24.0 ± 0.7 | 100 | 278.0 ± 23.5 |
| | for injections | 1 | −3 | −5 | −2 | 1 | 0 | | −1 |
| 15 | Without exposure | 105.4 ± 8.1 | 142.8 ± 10.3 | 194.2 ± 8.2 | 268.4 ± 19.8 | 636.7 ± 39.1 | 24 ± 0.9 | 100 | 274 ± 25.9 |

Note
BDF₁ mice, females with P-388 tumor

TABLE 2
GMDP-A effect on the primary tumor growth, metastasis and DDP therapeutic effect in mice with B16

| | | | | | | | | Average | | |
|---|---|---|---|---|---|---|---|---|---|---|
| | | Tumor volume, mm³ tumor growth inhibition (TGI), % | | | | | | life time, | | |
| Group | | tumor growth day | | | | | | days | Metastasis | |
| number | Therapeutic sequence | 7 | 9 | 12 | 14 | 16 | 19 | ILT, % | MF, % | MI, % |

Treatment at the early tumor progress (the 1^{st} day of tumor growth)

| | | | | | | | | | | |
|---|---|---|---|---|---|---|---|---|---|---|
| 1 | 5-fold GMDP-A | 11 ± 6 | 58 ± 11 | 177 ± 33 | 347 ± 47 | 522 ± 82 | 690 ± 208 | 21 ± 4 | 80 | 29 |
| | | 71 | 51 | 33 | 8 | 6 | 10 | 0 | | |
| 2 | 10-fold GMDP-A | 15 ± 6 | 56 ± 9 | 162 ± 21 | 298 ± 44 | 446 ± 53 | 894 ± 99 | 27 ± 4 | 40 | 81 |
| | | 62 | 52 | 38 | 21 | 20 | 16 | 29 | | |
| 3 | single DDP + 5-fold GMDP-A | 0 | 16 ± 5 | 61 ± 12 | 134 ± 19 | 304 ± 38 | 820 ± 126 | 29 ± 3 | 20 | 99 |
| | | 100 | 87 | 77 | 65 | 45 | 23 | 38 | | |
| 4 | single DDP + 10-fold GMDP-A | 0 | 9 ± 3 | 37 ± 11 | 105 ± 24 | 192 ± 37 | 554 ± 116 | 39 ± 10 | 20 | 99 |
| | | 100 | 93 | 86 | 72 | 65 | 48 | 86 | | |
| 5 | DDP | 3 ± 3 | 13 ± 5 | 54 ± 18 | 117 ± 20 | 232 ± 36 | 653 ± 176 | 28 ± 3 | 20 | 99 |
| | | 94 | 89 | 80 | 69 | 58 | 39 | 33 | | |

Treatment at the late tumor progress (the 5^{th} day of tumor growth)

| | | | | | | | | | | |
|---|---|---|---|---|---|---|---|---|---|---|
| 6 | 5-fold GMDP-A | n/d | 61 ± 9 | 173 ± 17 | 329 ± 41 | 391 ± 67 | 817 ± 234 | 18 ± 3 | 100 | 5 |
| | | | 48 | 34 | 13 | 30 | 23 | 0 | | |
| 7 | 10-fold GMDP-A | n/d | 66 ± 14 | 164 ± 29 | 269 ± 62 | 408 ± 79 | 882 ± 238 | 24 ± 4 | 100 | 8 |
| | | | 44 | 37 | 29 | 27 | 17 | 14 | | |
| 8 | single DDP + 5-fold GMDP-A | n/d | 50 ± 14 | 93 ± 21 | 191 ± 25 | 356 ± 53 | 728 ± 87 | 31 ± 4 | 20 | 99 |
| | | | 58 | 65 | 49 | 36 | 32 | 48 | | |
| 9 | single DDP + 10-fold GMDP-A | n/d | 58 ± 8 | 147 ± 25 | 241 ± 34 | 341 ± 39 | 842 ± 167 | 28 ± 6 | 30 | 83 |
| | | | 51 | 44 | 36 | 39 | 21 | 33 | | |
| 10 | DDP | n/d | 51 ± 8 | 163 ± 22 | 270 ± 45 | 424 ± 43 | 1010 ± 173 | 25 ± 3 | 100 | 11 |
| | | | 56 | 38 | 23 | 24 | 5 | 19 | | |

Control groups

| | | | | | | | | | | |
|---|---|---|---|---|---|---|---|---|---|---|
| 11 | 24 hours →10-fold water for | 36 ± 5 | 115 ± 8 | 250 ± 12 | 361 ± 35 | 553 ± 61 | 1059 ± 97 | 22 ± 5 | 100 | 0 |
| | injections | 8 | 3 | 5 | 5 | 0 | 0 | 5 | | |
| 12 | 5 days →10-fold water for injections | n/d | 119 ± 6 | 261 ± 18 | 359 ± 41 | 560 ± 46 | 1072 ± 89 | 21 ± 2 | 100 | 0 |
| | | | 0 | 0 | 5 | 0 | 0 | 0 | | |
| 13 | Without exposure | 39 ± 4 | 118 ± 10 | 262 ± 20 | 378 ± 39 | 557 ± 54 | 1066 ± 106 | 21 ± 3 | 100 | — |

Note:
BDF₁ mice, females with B16 tumor;
n/d—not defined

TABLE 3
GMDP-A impact on the chemotherapy with drugs: cisplatin, gemzar and
cyclophosphan

| | | Tumor growth | | | | | | | |
|---|---|---|---|---|---|---|---|---|---|
| | | inhibition (TGI), % | | | | | Average life | | |
| Group | | tumor growth day | | | | | time, days | Metastasis | |
| number | Therapeutic sequence | 7 | 12 | 14 | 19 | 21 | ILT, % | MF, % | MI, % |

Cisplatin + GMDP-A

| | | | | | | | | | |
|---|---|---|---|---|---|---|---|---|---|
| 1 | single DDP | 27 | 36 | 43 | 33 | 22 | 26 ± 1 | 100 | 40 |
| | | | | | | | 9 | | |
| 2 | single DDP + 21-fold | 80 | 78 | 81 | 78 | 76 | 29 ± 2 | 100 | 55 |
| | GMDP-A* | | | | | | 21 | | |
| 3 | single DDP + 21-fold | 73 | 73 | 75 | 81 | 80 | 34 ± 2 | 100 | 51 |
| | GMDP-A** | | | | | | 39 | | |

Gemzar + GMDP-A

| | | | | | | | | | |
|---|---|---|---|---|---|---|---|---|---|
| 4 | single Gemzar | 60 | 37 | 34 | 45 | 44 | 29 ± 2 | 100 | 69 |
| | | | | | | | 21 | | |
| 5 | single Gemzar + 21-fold | 94 | 96 | 93 | 87 | 88 | 38 ± 4 | 100 | 73 |
| | GMDP-A* | | | | | | 57 | | |
| 6 | single Gemzar + 21-fold | 89 | 84 | 79 | 76 | 84 | 34 ± 2 | 100 | 70 |
| | GMDP-A** | | | | | | 43 | | |

Cyclophosphan + GMDP-A

| | | | | | | | | | |
|---|---|---|---|---|---|---|---|---|---|
| 7 | single CP | 89 | 86 | 85 | 76 | 77 | 31 ± 2 | 100 | 89 |
| | | | | | | | 30 | | |
| 8 | single CP + 21-fold | 89 | 95 | 97 | 99 | 99 | 39 ± 2 | 100 | 88 |
| | GMDP-A* | | | | | | 61 | | |
| 9 | single CP + 21-fold | 90 | 96 | 97 | 94 | 91 | 37 ± 3 | 100 | 90 |
| | GMDP-A** | | | | | | 54 | | |

Control groups

| | | | | | | | | | |
|---|---|---|---|---|---|---|---|---|---|
| 10 | 10-fold water for injections | — | — | — | — | — | 24 ± 3 | 100 | −3 |
| | | | | | | | 0 | | |
| 11 | Without exposure | — | — | — | — | — | 24 ± 1 | 100 | |

Note:
BDF1 mice, females with P-388 tumor
*GMDP-A subcutaneous injection near the tumor node
**GMDP-A subcutaneous injection on the opposite side of the tumor node

TABLE 4
Comparison of GMDP-A lyothilisate and final pharmaceutical form for mice with P388 lymphoplastic leukemia

| | | Tumor volume, mm³ tumor growth inhibition | | | | | | |
|---|---|---|---|---|---|---|---|---|
| | | (TGI), % | | | | Average life | Metastasis | |
| Group | | tumor growth day | | | | time, days | | Metastasis |
| number | Therapeutic sequence | 8 | 12 | 16 | 20 | ILT, % | MF, % | inhibition index, % |

GMDP-A lyophilisate

| | | | | | | | | |
|---|---|---|---|---|---|---|---|---|
| 1 | 21-fold GMDP-A | 48 ± 4 | 101 ± 7 | 277 ± 13 | 713 ± 44 | 29.0 ± 1.2 | 100 | 214.0 ± 16.5 |
| | lyothilisate | 44 | 38 | 23 | 31 | 23 | | 15 |
| 2 | DDP + 21-fold GMDP-A | 0.5 ± 0.5 | 2 ± 1 | 7 ± 2 | 213 ± 15 | 35.0 ± 2.2 | 100 | 45.0 ± 6.6 |
| | lyothilisate | 99 | 99 | 98 | 79 | 44 | | 82 |

GMDP-A final pharmaceutical form

| | | | | | | | | |
|---|---|---|---|---|---|---|---|---|
| 3 | 21-fold GMDP-A final | 51 ± 5 | 103 ± 10 | 275 ± 27 | 741 ± 33 | 30.0 ± 1.3 | 100 | 199.0 ± 53.0 |
| | pharmaceutical form | 40 | 37 | 24 | 29 | 26 | | 21 |
| 4 | single DDP + 21-fold | 1.0 ± 0.7 | 1.0 ± 0.7 | 7 ± 3 | 182 ± 12 | 33.0 ± 1.4 | 100 | 51.7 ± 6.8 |
| | GMDP-A final | 99 | 99 | 98 | 82 | 37 | | 79 |
| | pharmaceutical form | | | | | | | |

Control

| | | | | | | | | |
|---|---|---|---|---|---|---|---|---|
| 5 | DDP | 29.4 ± 5.3 | 53.6 ± 5.4 | 96.3 ± 5.1 | 440 ± 26.7 | 30.0 ± 2.8 | 100 | 172.0 ± 14.4 |
| | | 65 | 67 | 73 | 58 | 22 | | 32 |
| 6 | 10-fold water for injections | 83.5 ± 7.4 | 204.2 ± 11.9 | 423.1 ± 36.6 | 945 ± 53 | 23.0 ± 1.0 | 100 | 283.0 ± 17.6 |
| | | 1 | −26 | −17 | 9 | −3 | | −12 |
| 7 | Without exposure | 84.5 ± 7.5 | 162.5 ± 15.2 | 361.6 ± 33.5 | 1037 ± 84 | 24.0 ± 1.0 | 100 | 252.0 ± 49 |

Note
BDF₁ mice, females with P-388 tumor

1. Method for treating malignant hematological diseases or melanoma in subjects by applying one or more cytostatics impacting DNA in combination with N-acetyl-D-glucosaminyl-β-(1-4)-N-acetylmuramyl-L-alanyl-D-glutamic acid (GMDP-A) according to the following therapeutic sequence for subjects:
Intravenous injection of ¼ to ½ standard therapeutic dose of the cytostatic selected for this type of subjects;
Then, after cytostatic administration, the first injection of N-acetyl-D-glucosaminyl-β-(1-4)-N-acetylmuramyl-L-alanyl-D-glutamic acid (GMDP-A) in effective amount set forth for these subjects;
GMDP-A repeated injections in effective amount set forth for selected subjects.

2. Method according to claim 1, characterized in that an animal or a human being is the therapy subject

3. Method according to claim 1, characterized in that the first and repeated injections are made subcutaneously.

4. Method according to claim, characterized in that the first GMDP-A injection is made in an hour after cytostatic administration while repeated subcutaneous injections of GMDP-A are made once per day within 4 days to 20 days.

5. A method according to claim 2, characterized in that the first GMDP-A injection is made in an hour after cytostatic administration while repeated subcutaneous injections of GMDP-A are made once per day within 4 days to 20 days.

6. A method according to claim 3, characterized in that the first GMDP-A injection is made in an hour after cytostatic administration while repeated subcutaneous injections of GMDP-A are made once per day within 4 days to 20 days.
